(19) United States
(12) Patent Application Publication
(10) Pub. No.: US 2012/0189585 A1
(43) Pub. Date: Jul. 26, 2012

(54) METHOD AND COMPOSITION FOR RESTORATION OF AGE-RELATED TISSUE LOSS IN THE FACE OR SELECTED AREAS OF THE BODY

(75) Inventors: Vincent Giampapa, Montclair, NJ (US); Robin L. Smith, New York, NY (US)

(73) Assignee: NEOSTEM, INC., New York, NY (US)

(21) Appl. No.: 13/318,524

(22) PCT Filed: May 4, 2010
(86) PCT No.: PCT/US10/33463

§ 371 Date: Apr. 16, 2012

Related U.S. Application Data
(60) Provisional application No. 61/175,275, filed on May 4, 2009.

(51) Int. Cl. A61K 35/12; A61K 35/14; A61P 17/00; A61K 35/28

(52) U.S. Cl. 424/93.7

(57) ABSTRACT

This application relates to stem cell compositions and methods for restoring age-related tissue loss in the face and other selected areas of the body. In a first embodiment, a composition includes stem cells and hyaluronic acid as a carrier wherein the stem cells are peripheral blood stem cells, bone marrow-derived blood stem cells, or mesenchymal stem cells.

### RELATED APPLICATIONS

This application claims the benefit of priority to U.S. Provisional Application No. 61/175,275, filed 4 May 2009, which is herein incorporated by reference in its entirety.

### FIELD OF THE DISCLOSURE

The present invention relates to methods of restoration of age-related tissue loss in the face or selected areas of the body. More specifically, the method injects autologous stem cells, which restores age-related tissue loss in the face or selected areas of the body.

### BACKGROUND OF THE DISCLOSURE

Skin is composed of the epidermis and the dermis. Below these layers lies the hypodermis, which is not usually classified as a layer of skin. The hypodermis is also commonly referred to as subcutaneous fat layer, or subcutaneous tissue. The outermost epidermis is made up of stratified squamous epithelium with an underlying basement membrane. It contains no blood vessels, and is nourished by diffusion from the dermis. The main type of cells which make up the epidermis are keratinocytes. Also present are melanocytes and langerhans cells. This layer of skin is responsible for keeping water in the body and keeping harmful chemicals and pathogens out.

The dermis lies below the epidermis and contains a number of structures including blood vessels, nerves, hair follicles, smooth muscle, glands and lymphatic tissue. The dermis (or corium) is typically 3-5 mm thick and is the major component of human skin. It is composed of a network of connective tissue, predominantly collagen fibrils providing support and elastic tissue providing flexibility. The main cell types are fibroblasts, adipocytes and macrophages. The hypodermis lies below the dermis. Its purpose is to attach the skin to underlying bone and muscle as well as supplying it with blood vessels and nerves. It is made up of loose connective tissue and elastin. The main cell types are fibroblasts, macrophages and adipocytes. The hypodermis contains 50% of body fat. Fat serves as padding and insulation for the body.

Facial aging occurs as the result of several factors: inherent changes within the skin, effects of gravity, facial muscles acting on the skin (dynamic lines), soft tissue loss or shift and bone loss and loss of tissue elasticity. The skin ages when the epidermis begins to thin, causing the junction with the dermis to flatten. Collagen decreases as a person ages and the bundles of collagen, which gives the skin turgor, become looser and lose strength. When the skin loses elasticity, it is less able to resist stretching. Coupled with gravity, muscle pull and tissue changes, the skin begin to wrinkle Water loss and breakdown of bonds between cells also reduces the barrier function of the skin, which can cause the skin's pore size to increases.

As a person ages, the face loses volume, soft tissue, and fat. The appearance of jowls and folds are usually caused by the drooping of facial tissues and folding of areas where the muscles below are attached to the skin. As part of the reduction in soft tissue the face gets more hollow.

More specifically, in various facial areas, such as forehead, eyes, nose, midface and lower face, changes relating to aging have been well documented. In the forehead area, the forehead and brow droop over time, which lowers the eyebrows and causes the upper eyelid skin to bunch. Forehead lines appear when one tries to hold the brows and eyelids up to counteract these changes. It is well known that the eyes are often the first facial feature to show signs of aging. Skin changes around the eyes occur earlier than in the rest of the face since the skin is thinner around the eyes. The skin here contains fewer glands and is subjected to constant blinking, squinting, rubbing, and pulling. The midface ages when the cheeks begin to droop, causing nasolabial folds. Nasolabial folds are the lines that run from the sides of the nose to the corners of the mouth. These folds have been treated with facial fillers. In the nose area, as a person ages, the nose elongates. Common causes of elongation are thinning of the soft tissue and loss of elasticity, which causes “drooping of the tip” and unmasking of the bone, creating a new hump. In the lower face area, as the face ages, facial tissues descend. This results in the so-called “laugh lines”. Folds and lines in this area have been treated with facial fillers. Further down on the face, the corners of the mouth may droop and descent of the jowls can create folds often referred to as “marionette” lines. Furthermore, jowls form when the cheeks sag around a fixed point along the jaw where the facial muscles attach to the jawbone. The facial muscles continue down into the neck as a sheet called the platysma muscle. This muscle often gaps in the center of the neck, creating two bands.

Various injectables have been used for restoring tissue loss in the face. Since the 1980s, injectable collagen has been used as a soft-tissue filler to fill wrinkles, lines and scars on the face. Collagen is a naturally occurring protein that supports various parts of the body including skin, tendons and ligaments. Fat injections have been used for years to add volume, fill wrinkles, lines and enhance the lips. Fat injections involve taking fat from one part of the patient's body (abdomen, thighs or buttocks) and reinjecting it beneath the facial skin. Botulinum toxins have been used for neck spasms, cranial nerve disorders and eye spasms. With the recent FDA approval of Botox for cosmetic use in the glabellar region, the drug is used to smooth wrinkles. When injected into facial muscles botulinum toxins block nerve impulses, temporarily paralyzing muscles and smoothing wrinkles.

Hyaluronic acid is one of most commonly used cosmetic dermal filler which adds volume to minimize wrinkles and lines. Hyaluronic acid is a linear polysaccharide that exists naturally in all living organisms and is a universal component of the extra-cellular spaces of body tissues. The identical structure of hyaluronic acid in all species and tissues makes this polysaccharide an ideal substance for use as a bio-material in health and medicine. Hyaluronic acid is present in many places in the human body. It gives volume to the skin, shape to the eyes and elasticity to the joints. The highest concentrations are found in connective tissues, and most hyaluronic acid (about 56%) is found in the skin.

Various forms of hyaluronic acid are provided commercially by a number of manufacturers. The most commonly used hyaluronic acid is the non-animal stabilized hyaluronic acid (NASHA) in a clear gel form, produced by bacterial fermentation from streptococci bacteria. Different from animal derived hyaluronic acid, the non-animal derived hyaluronic acid is free from animal proteins. This limits the risk of animal based disease transmissions or development of allergic reactions to animal proteins. The most known non-animal stabilized hyaluronic acid is manufactured by Q-med, Seminariegatan, Uppsala, and commercially available under the tradename Restylane®. Since its commercialization in 1996, it is estimated that over 2,500,000 treatments have been carried out worldwide. Other non-animal stabilized hyaluronic acid products include Perlane® from Q-med, which has larger particles than Restylane®, and Captique™ from Genzyme Corporation. Another commonly used filler is hyaluronan manufactured by Genzyme Corporation and commercially available under the tradename Hylaform Plus. Hylaform Plus is a sterile, nonpyrogenic, viscoelastic, clear, colorless, transparent gel implant composed of cross-linked molecules of hyaluronan.

Although hyaluronic acid and derivatives are the most commonly used dermal fillers, they have limited viability. The re-injection is needed every 4 to 12 months, or even shorter.

It has been reported that the skin can be injected with growth factors in order to restore the appearance of the skin by increasing the amount of fat in the dermis. Pre-adipocyte stimulation with specific growth factors induces accelerated differentiation of uncommitted cells into fat cells resulting in local augmentation of fat at the injection sites. These growth factors include insulin, insulin-like growth factor, triiodothyronine (T3), thyroxine (T4), and retinoic acid.

As taught in U.S. Pat. No. 7,414,021. which is hereby incorporated by reference in its entirety, an injectable composition can comprise hyaluronic acid as a carrier and at least one growth factor, which acts as a signaling molecule between cells and promotes differentiation and maturation of the cells. Such growth factors act similarly to hormones. Suitable hormones useful as part of an injectable composition are thyroid hormones and estrogens. U.S. Pat. No. 7,414,021 states that a number of different growth factors may be used to stimulate pre-adipocytes and induce their accelerated differentiation into mature adipocytes at the injection sites; whereas, specific growth factors, such as estrogen, stimulates elastin and collagen production upon injection into the dermis. The combination of the effects achieved in the dermis layer and the underlying hypodermis layer results in the restoration of age-related tissue loss at the treatment sites.

Although various injectables have been developed and used clinically for restoration of age-related tissue loss in the face over the passed 50 years, due to various limitations in the materials or compatibility with the tissues, the long term effect in restoring the tissue loss is limited. It is desirable to develop improved injectables and treatment methods to enhance the overall effects in restoration of age-related tissue loss in the face or selected areas of the body, such as neck and hands. The present invention provides an improved injectable and method of delivering them. The improved injectable comprises autologous stem cells, though stem cells from sources other than the individual may be used. A novel method of delivering the stem cells comprises use of microabrasion therapy.

The use of stem cells and stem cell derivatives has gained increased interest in medical research, particularly in the area of providing reagents for treating tissue damage either as a result of genetic defects, injuries, and/or disease processes. Ideally, cells that are capable of differentiating into the affected cell types could be transplanted into a subject in need thereof, where they would interact with the organ microenvironment and supply the necessary cell types to repair the injury. The applicants recognize for the first time that such stem cells can be used for the restoration of age-related tissue loss in the face and selected other areas of the body.

The applicants also recognized the desire to create an environment for the injected stem cells to take hold and flourish. Accordingly, the applicants perform microdermabrasion on the skin prior to the injection of stem cells.

Throughout this description, including the foregoing description of related art, any and all publicly available documents described herein, including any and all U.S. patents, are specifically incorporated by reference herein in their entirety. The foregoing description of related art is not intended in any way as an admission that any of the documents described therein, including pending United States patent applications, are prior art to embodiments of the present disclosure. Moreover, the description herein of any disadvantages associated with the described products, methods, and/or apparatus, is not intended to limit the disclosed embodiments. Indeed, embodiments of the present disclosure may include certain features of the described products, methods, and/or apparatus without suffering from their described disadvantages.

### SUMMARY OF THE DISCLOSURE

The present invention is directed to a treatment method of restoring age-related tissue loss in the face or selected areas of the body of a person. In one embodiment, the treatment method comprises the steps of providing a composition comprising stem cells and a carrier; and injecting the composition into the dermis, or the hypodermis at one or more areas of the face, or selected areas of the body of a person to stimulate collagen, elastin, or fat cell production, thereby restoring age-related tissue loss in the face, or the selected areas of the body of the person.

In another embodiment of the invention, the treatment method further comprises initially repeating the treatment session about twice, with a time interval between two sessions of about one week.

In yet another embodiment of the invention, the treatment method further comprises repeating the treatment session once every two months for one year.

In another embodiment of the invention, the composition may comprise hyaluronic acid as a carrier. Additionally, the treatment method may also be combined with injections of hyaluronic acid combined with certain growth factors. The hyaluronic acid is absorbed in the dermis or the hypodermis with time, thereby providing time release of the growth factor in the composition into the dermis or the hypodermis. The growth factors can be used for the method of the present invention include insulin, an insulin-like growth factor, a thyroid hormone, a fibroblast growth factor, an estrogen, retinoic acid, or combination thereof.

In another embodiment of the invention, the injectible composition may comprise adipocytes. The adipocytes can function in their traditional role in face lifts by acting as padding; however, the adipocytes are also capable of creating an environment that allows the injected pluripotent cells themselves to commit to becoming adipocytes.

In a further embodiment, the present invention is directed to a method for preparing the skin before the introduction of the stem cells, which allows the stem cells to take hold and flourish after being injected into the skin. The skin is treated by performing microdermabrasion to promote cellular repair, which creates an environment that allows the injected stem cells to thrive.

According to some embodiments, a composition is provided comprising stem cells and hyaluronic acid as a carrier, wherein said stem cells are peripheral blood stem cells, bone marrow-derived stem cells, or mesenchymal stem cells. Preferably, the stem cell are autologous stem cells.

According to some embodiments, a treatment method is provided for restoring tissue volume in the face or selected areas of the body of a person comprising administering to a human subject a composition comprising stem cells and hyaluronic acid as a carrier, wherein said stem cells are peripheral blood stem cells, bone marrow-derived stem cells, or mesenchymal stem cells, and wherein said composition is administered via injection into the dermis or the hypodermis at one or more areas of the face, thereby restoring tissue volume said selected areas of the body of said person. Preferably, the stem cell are autologous stem cells.

In some embodiments, the composition further comprises adipocytes or preadipocytes.

In some embodiments, the composition further comprises insulin, an insulin-like growth factor, a thyroid hormone, a fibroblast growth factor, an estrogen, retinoic acid, or combination thereof. The thyroid hormone may be liothyronine sodium, levothyroxine sodium, or combination thereof.

In some embodiments, the one or more areas of the face is the peri-orbital area, the lips, the malar area, the nasolabial folds, the labio-mandibular folds, the neck, or the hands.

In some embodiments, the stem cell composition of the present disclosure further comprises retinoic acid. In some embodiments, the stem cell composition of the present disclosure further comprises deuterium reduced water.

### BRIEF DESCRIPTION OF THE DRAWINGS

For a better understanding of the present invention, reference is made to the following description, taken in conjunction with the accompanying drawings, in which like reference characters refer to like parts throughout.

FIG. 1 shows the in vitro differentiation of human mesenchymal stem cells into preadipocytes or adipocytes.

### DETAILED DESCRIPTION OF THE INVENTION

For the purposes of promoting an understanding of the principles of the invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

### Injectable Compositions

In one aspect, the present invention provides an injectable composition comprising stem cells (e.g., pluripotent stem cells) for injection into the dermis or the hypodermis (subcutaneous tissue) to restore age-related tissue loss in the face, and selected areas of the body, such as neck and hands. According to some embodiments, the stem cells are autologous stem cells. According to some embodiments, the stem cells are autologous peripheral blood stem cells. According to some embodiments, the stem cells are autologous mesenchymal stem cells (MSCs). According to some embodiments, the stem cells are autologous Very Small Embryonic-Like (VSEL) stem cells.

The stem cells of the present invention may be formulated into an injectable subcutaneous formulation using any usable carrier (e.g., pharmaceutically acceptable carrier). Saline is an appropriate carrier, as is any sterile physiologic buffer.

### Stem Cells

The stem cells according to the present invention, which includes pluripotent stem cells, are isolated by methods that are well-known in the art. Any type of uncommitted pluripotent or totipotent cell may be used as part of this invention, including stem cells from both embryonic and adult sources. Stem cells from a number of different tissues have been isolated for use in regenerative medicine. For example, U.S. Pat. No. 5,750,397 to Tsukamoto et al., incorporated herein by reference in its entirety, discloses the isolation and growth of human hematopoietic stem cells that are reported to be capable of differentiating into lymphoid, erythroid, and myelomonocytic lineages. U.S. Pat. No. 5,736,396 to Bruder et al., incorporated herein by reference in its entirety, discloses methods for lineage-directed differentiation of isolated human mesenchymal stem cells under the influence of appropriate growth and/or differentiation factors. The derived cells can then be introduced into a host for mesenchymal tissue regeneration or repair.

The invention contemplates the use of a variety of stem cells. For example, mesenchymal stem cells (MSCs) are one such cell type. MSCs have been shown to have the potential to differentiate into several lineages including bone (Haynesworth et al. (1992) 13 Bone 81-88), cartilage (Mackay et al. (1998) 4 Tissue Eng 41 5-28; Yoo et al. (1998) 80 J Bone Joint Surg Am 745-57), adipose tissue (Pittenger et al. (2000) 251 Curr Top Microbiol Immunol-11), tendon (Young et al. (1998) 16 J Orthop Res 406-13), muscle, and stroma (Caplan et al. (2001) 7 Trends Mol Med 259-64). Very small embryonic-like (VSEL) stem cells identified by Ratajczak (WO 2007/067280), incorporated herein by reference in its entirety, may be used.

Homogeneous human mesenchymal stem cell compositions are provided which serve as the progenitors for all mesenchymal cell lineages. MSCs are identified by specific cell surface markers which are identified with unique monoclonal antibodies. The homogeneous MSC compositions are obtained by positive selection of adherent marrow or periosteal cells which are free of markers associated with either hematopoietic cell or differentiated mesenchymal cells. These isolated mesenchymal cell populations display epitopic characteristics associated with only mesenchymal stem cells, have the ability to regenerate in culture without differentiating, and have the ability to differentiate into specific mesenchymal lineages when either induced in vitro or placed in vivo at the site of damaged tissue.

In order to obtain subject human mesenchymal stem cells, it is necessary to isolate rare pluripotent mesenchymal stem cells from other cells in the bone marrow or other MSC source. Bone marrow cells may be obtained from iliac crest, femora, tibiae, spine, rib or other medullary spaces. Other sources of human mesenchymal stem cells include embryonic yolk sac, placenta, umbilical cord, fetal and adolescent skin, and blood.

The method of their isolation comprises the steps of providing a tissue specimen containing mesenchymal stem cells, adding cells from the tissue specimen to a medium which contains factors that stimulate mesenchymal stem cell growth without differentiation and allows, when cultured, for the selective adherence of only the mesenchymal stem cells to a substrate surface, culturing the specimen-medium mixture, and removing the non-adherent matter from the substrate surface.

In another aspect, the present invention relates to a medium for isolating human mesenchymal stem cells from a tissue specimen, wherein the medium is comprised of factors which stimulate mesenchymal stem cell growth without differentiation and allows, when cultured, for the selective adherence of only the mesenchymal stem cells to a substrate surface.

In an additional aspect, the invention is directed to a method for culture-expanding the isolated and/or purified mesenchymal stem cells, e.g., marrow, blood, periosteum or dermis derived. The method comprises the steps of providing a tissue specimen containing mesenchymal stem cells, adding cells from the specimen to a medium which contains factors that stimulate mesenchymal stem cell growth without differentiation and allows, when cultured, for the selective adherence of only the mesenchymal stem cells to a substrate surface, culturing the tissue specimen-medium mixture, removing the non-adherent matter from the substrate surface by replacing the medium with a fresh medium of the same composition, and, allowing the isolated adherent mesenchymal stem cells to culture-expand. It has been found that the differential and/or reparative potential of the culture-expanded mesenchymal stem cells is maintained during culture expansion under specific conditions. Method for obtaining human mesenchymal stem cells is described in U.S. Pat. No. 5,486,359, incorporated herein by reference in its entirety.

Another population of cells, multipotent adult progenitor cells (MAPCs), has also been purified from bone marrow (BM; Reyes et al. (2001) 98 Blood 25 2615-2625; Reyes & Vetfaillie (2001) 938 Ann NY Acad Sci 231-235). These cells have been shown to be capable of expansion in vitro for more than 100 population doublings without telomere shortening or the development of karyotypic abnormalities. MAPCs have also been shown to be able to differentiate under defined culture conditions into various mesenchymal cell 30 types (e.g., osteoblasts, chondroblasts, adipocytes, and skeletal myoblasts), endothelium, neuroectoderm cells, and more recently, into hepatocytes (Schwartz et al. (2000) 109 J Clin Invest 1291-1302).

Additionally, hematopoietic stem cells (HSCs) have been reported to be able to differentiate into numerous cell types. BM hematopoietic stem cells have been reported to be able to ‘transdifferentiate’ into cells that express early heart (Orlic et al. (2003) 7 Pediatr Transplant 86-88; Makino et al. (1999) 103 J Clin Invest 697-705), skeletal muscle (Labarge & Blau (2002) 111 Cell 589-601; Corti et al. (2002) 277 Exp Cell Res 74-85), neural (Sanchez-Ramos (2002) 69 Neurosci Res 880-893), liver (Petersen et al. (1999) 284 Science 1168-1170), or pancreatic cell (Lanus et al. (2003) 111 J Clin Invest 843-850; Lee & Stoffel (2003) 111 J Clin Invest 799-801) markers. In vivo experiments in humans also demonstrated that transplantation of CD34+ peripheral blood (PB) stem cells led to the appearance of donor-derived hepatocytes (Korbling et al. (2002) 346 N Engl J Med 738-746), epithelial cells (Korbling et al. (2002) 346 N Engl J Med 738-746), and neurons (Hao et al. (2003) 12 J Hematother Stem Cell Res 23-32). Additionally, human BM-derived cells have been shown to contribute to the regeneration of infarcted myocardium (Stamm et al., (2003) 361 Lancet 45-46).

### Stem Cell Enrichment or Sorting

Stem cells may be sorted according to cell surface markers that are associated with stem cells. Since it is one embodiment of the invention to enrich for stem cells, useful markers for cell sorting need not be exclusively expressed in stem cells. A cell marker which is not exclusively expressed in stem cell will nevertheless have utility in enriching for stem cells. It should noted also that markers of differentiated cells are also useful in the methods of the invention because these markers may be used, for example, to selectively remove differentiated cells and thus enrich stem cells in the remaining cell population. Markers, cell surface or otherwise, which may be used in any of the processes of the invention include, at least, the following:

| Marker | Cell Type | Significance |
|---|---|---|
| | | Blood Vessel |
| Fetal liver | Endothelial | Cell-surface receptor protein that identifies |
| kinase-1 (Flk1) | | endothelial cell progenitor; marker of cell-cell contacts |
| | | Bone |
| Bone-specific | Osteoblast | Enzyme expressed in osteoblast; activity |
| alkaline | | indicates bone formation |
| phosphatase (BAP) | | |
| | | Bone Marrow and Blood |
| Bone morphogenetic | Mesenchymal | Important for the differentiation of committed |
| protein receptor | stem and | mesenchymal cell types from mesenchymal stem |
| (BMPR) | progenitor cells | and progenitor cells; BMPR identifies early |
| | | mesenchymal lineages (stem and progenitor cells) |
| CD34 | Hematopoietic | Cell-surface protein on bone marrow cell, |
| | stem cell (HSC), | indicative of a HSC and endothelial progenitor; |
| | satellite, | CD34 also identifies muscle satellite, a muscle stem cell |
| | endothelial progenitor | |
| CD34⁺, Sca1⁺, | Mesencyhmal | Identifies MSCs, which can differentiate into |
| Lin⁻ profile | stem cell (MSC) | adipocyte, osteocyte, chondrocyte, and myocyte |
| CD38 | Absent on HSC | Cell-surface molecule that identifies WBC |
| | Present on | lineages. Selection of CD34+/CD38− cells |
| | WBC lineages | allows for purification of HSC populations |
| c-Kit | HSC, MSC | Cell-surface receptor on BM cell types that |
| | | identifies HSC and MSC; binding by fetal calf |
| | | serum (FCS) enhances proliferation of ES cells, |
| | | HSCs, MSCs, and hematopoietic progenitor cells |
| Colony-forming | HSC, MSC | Progenitor CFU assay detects the ability of a |
| unit (CFU) | | single stem cell or progenitor cell to give rise to |
| | | one or more cell lineages, such as red blood cell |
| | | (RBC) and/or white blood cell (WBC) lineages |
| Fibroblast colony- | Bone marrow | An individual bone marrow cell that has given |
| forming unit | fibroblast | rise to a colony of multipotent fibroblastic cells; |
| (CFU-F) | | such identified cells are precursors of |
| | | differentiated mesenchymal lineages |
| Hoechst dye | Absent on HSC | Fluorescent dye that binds DNA; HSC extrudes |
| | | the dye and stains lightly compared with other cell types |
| KDR | Hematopoietic | VEGF-receptor 2. Present in hematopoietic stem |
| | stem cell and | cells and progenitor cells. |
| | progenitor cell. | |
| Leukocyte | WBC | Cell-surface protein on WBC progenitor |
| common antigen | | |
| (CD45) | | |
| Lineage surface | HSC, MSC | Thirteen to 14 different cell-surface proteins that are |
| antigen (Lin) | Differentiated | markers of mature blood cell lineages; detection |
| | RBC and WBC | of Lin-negative cells assists in the purification of |
| | lineages | HSC and hematopoietic progenitor populations |
| Muc-18 (CD146) | Bone marrow | Cell-surface protein (immunoglobulin |
| | fibroblasts, | superfamily) found on bone marrow fibroblasts, |
| | endothelial | which may be important in hematopoiesis; a |
| | | subpopulation of Muc-18+ cells are |
| | | mesenchymal precursors |
| Stem cell antigen | HSC, MSC | Cell-surface protein on bone marrow (BM) cell, |
| (Sca-1) | | indicative of HSC and MSC Bone Marrow and Blood cont. |
| Stro-1 antigen | Stromal | Cell-surface glycoprotein on subsets of bone |
| | (mesenchymal) | marrow stromal (mesenchymal) cells; selection |
| | precursor cells, | of Stro-1+ cells assists in isolating mesenchymal |
| | hematopoietic cells | precursor cells, which are multipotent cells that |
| | | give rise to adipocytes, osteocytes, smooth |
| | | myocytes, fibroblasts, chondrocytes, and blood cells |
| Thy-1 | HSC, MSC | Cell-surface protein; negative or low detection is |
| | | suggestive of HSC |
| CD14 | monocytes | Monocyte differentiation to dendritic cells. |
| Platelet | neutrophils, | Endothelial cell adhesion |
| Endothelial Cell | macrophages | |
| Adhesion Molecule | | |
| (PECAM-1 or CD31) | | |
| CD73 | Lymphocyte | Lymphocyte maturation cell marker |
| | | Fat |
| Adipocyte lipid- | Adipocyte | Lipid-binding protein located specifically in adipocyte |
| binding protein | | |
| (ALBP) | | |
| Fatty acid | Adipocyte | Transport molecule located specifically in adipocyte |
| transporter (FAT) | | |
| Adipocyte lipid- | Adipocyte | Lipid-binding protein located specifically in adipocyte |
| binding protein | | |
| (ALBP) | | |
| | | Liver |
| B-1 integrin | Hepatocyte | Cell-adhesion molecule important in cell-cell |
| | | interactions; marker expressed during development of liver |
| | | Nervous System |
| CD133 | Neural stem | Cell-surface protein that identifies neural stem |
| | cell, HSC | cells, which give rise to neurons and glial cells |
| Glial fibrillary | Astrocyte | Protein specifically produced by astrocyte |
| acidic protein | | |
| (GFAP) | | |
| O4 | Oligodendrocyte | Cell-surface marker on immature, developing oligodendrocyte |
| CD166 | Neural cell | Neural cell marker; activated T-cells |
| | marker | |
| | | Pancreas |
| Cytokeratin 19 | Pancreatic | CK19 identifies specific pancreatic epithelial |
| (CK19) | epithelium | cells that are progenitors for islet cells and ductal cells |
| Nestin | Pancreatic | Structural filament protein indicative of |
| | progenitor | progenitor cell lines including pancreatic |
| | | Pluripotent Stem Cells |
| Alkaline | Embryonic stem | Elevated expression of this enzyme is associated |
| phosphatase | (ES) embryonal | with undifferentiated pluripotent stem cell (PSC) |
| | carcinoma (EC) | |
| Alpha-fetoprotein | Endoderm | Protein expressed during development of |
| (AFP) | | primitive endoderm; reflects endodermal |
| | | differentiation Pluripotent Stem Cells |
| Bone | Mesoderm | Growth and differentiation factor expressed |
| morphogenetic | | during early mesoderm formation and differentiation |
| protein-4 | | |
| Brachyury | Mesoderm | Transcription factor important in the earliest |
| | | phases of mesoderm formation and |
| | | differentiation; used as the earliest indicator of |
| | | mesoderm formation |
| Cluster | ES, EC | Surface receptor molecule found specifically on PSC |
| designation 30 | | |
| (CD30) | | |
| Cripto (TDGF-1) | ES, | Gene for growth factor expressed by ES cells, |
| | cardiomyocyte | primitive ectoderm, and developing cardiomyocyte |
| GATA-4 gene | Endoderm | Expression increases as ES differentiates into endoderm |
| GCTM-2 | ES, EC | Antibody to a specific extracellular-matrix |
| | | molecule that is synthesized by undifferentiated PSCs |
| Genesis | ES, EC | Transcription factor uniquely expressed by ES |
| | | cells either in or during the undifferentiated state |
| | | of PSCs |
| Germ cell nuclear | ES, EC | Transcription factor expressed by PSCs |
| factor | | |
| Hepatocyte | Endoderm | Transcription factor expressed early in endoderm formation |
| nuclear factor-4 | | |
| (HNF-4) | | |
| Nestin | Ectoderm, | Intermediate filaments within cells; characteristic |
| | neural and | of primitive neuroectoderm formation |
| | pancreatic | |
| | progenitor | |
| Neuronal cell- | Ectoderm | Cell-surface molecule that promotes cell-cell |
| adhesion molecule | | interaction; indicates primitive neuroectoderm formation |
| (N-CAM) | | |
| Oct-4 | ES, EC | Transcription factor unique to PSCs; essential for |
| | | establishment and maintenance of undifferentiated PSCs |
| Pax6 | Ectoderm | Transcription factor expressed as ES cell |
| | | differentiates into neuroepithelium |
| Stage-specific | ES, EC | Glycoprotein specifically expressed in early |
| embryonic | | embryonic development and by undifferentiated PSCs |
| antigen-3 (SSEA-3) | | |
| Stage-specific | ES, EC | Glycoprotein specifically expressed in early |
| embryonic | | embryonic development and by undifferentiated PSCs |
| antigen-4 (SSEA-4) | | |
| Stem cell factor | ES, EC, HSC, MSC | Membrane protein that enhances proliferation of |
| (SCF or c-Kit | | ES and EC cells, hematopoietic stem cell |
| ligand) | | (HSCs), and mesenchymal stem cells (MSCs); |
| | | binds the receptor c-Kit |
| Telomerase | ES, EC | An enzyme uniquely associated with immortal |
| | | cell lines; useful for identifying undifferentiated PSCs |
| TRA-1-60 | ES, EC | Antibody to a specific extracellular matrix |
| | | molecule is synthesized by undifferentiated PSCs |
| TRA-1-81 | ES, EC | Antibody to a specific extracellular matrix |
| | | molecule normally synthesized by undifferentiated PSCs |
| Vimentin | Ectoderm, | Intermediate filaments within cells; characteristic |
| | neural and | of primitive neuroectoderm formation |
| | pancreatic | |
| | progenitor | |
| | | Skeletal Muscle/Cardiac/Smooth Muscle |
| MyoD and Pax7 | Myoblast, | Transcription factors that direct differentiation of |
| | myocyte | myoblasts into mature myocytes |
| Myogenin and | Skeletal | Secondary transcription factors required for |
| MR4 | myocyte | differentiation of myoblasts from muscle stem cells |
| CD36 (FAT) | Cardiac cell | Integral membrane protein |
| | marker | |
| | | Progenitor Cell Marker |
| CD29 | | Late antigen receptor involved in cell-cell adhesions |

The pattern of markers express by stem cells may also be used to sort and categorize stem cells with greater accuracy. Any means of characterizing, including the detection of markers or array of markers, may be used to characterized and/or identify the cells obtained through the embodiments disclosed herein. For example, certain cell types are known to express a certain pattern of markers, and the cells collected by the processes described herein may be sorted on the basis of these known patterns. The table that follows provides examples of the identifying pattern or array of markers that may be expressed by certain cell types.

| Cell Type | Markers |
|---|---|
| Hematopoietic stem cell | C34, CD45, CXCR4 |
| Endothelial Progenitors | CD34, CD73, CD133, CXCR4, KDR, |
| Cells | anti-M IgG |
| Mesenchymal Stem Cells | CD34, CD45, CD90, CD105, CD106, CD44 |
| Very Small Embryonic | CD34⁺/lin⁻/CD45⁻ or Sca-1⁺/ |
| Like Cell. (VSEL) | lin⁻/CD45 |

In preferred embodiments, the stem cells are peripheral blood stem cells that express markers of pluripotency. That is, in response to appropriate differentiation signals, the stem cells differentiate along multiple pathways giving rise to many different phenotypes. According to another preferred embodiment, the stem cells may be induced to differentiate in vitro to cells that express at least one characteristic of a specialized tissue cell lineage (e.g., dermal or adipose cells). The fetal, non-neonatal or adult stem cells of the present embodiments may be induced to partially or totally differentiate into tissue cells having the features of tissue cells that include collagen, dermis, adipose, or muscle. For example, the stem cells may be cultured in vitro with growth factors that induce, partially (e.g., at least 30%, 40%, 50%, 60%, 70%, 80%, 90%, etc. of the cell population) or totally, the stem cells to differentiate into tissue cells (e.g., adipose tissue).

The stem cells or enriched populations of cells may be propagated or differentiated in vitro prior to injection or application to the skin of an individual. The stem cells may be differentiated by placing the cells under the influence of signals designed to induce specifically the foregoing phenotypes. Any method of subjecting the stem cells to such signals may include, but not limited to, transfection of stem cells with genes known to cause differentiation, and/or exposing the stem cells to differentiation agents. For example, the stem cells may be genetically modified either stably or transitorily to express exogenous genes or to repress the expression of endogenous genes. In such a manner, the differentiation of the stem cells may be controlled. As an alternative example, the stem cells, and colonies thereof, may be induced to differentiate along a predictable pathway through the use of media that favors the maintenance in culture of a phenotype.

In some instances, it would be beneficial to be able to isolate and purify stem and/or precursor cells from a subject that could be purified and/or manipulated in vitro before being reintroduced into the subject for treatment purposes. Growth factors have a positive effect on cell growth and cellular differentiation. The growth factors suitable for the purpose of the present invention include, but are not limited to, insulin, insulin-like growth factor, thyroid hormone, fibroblast growth factor, estrogen, retinoic acid, or combination thereof.

### Hyaluronic Acid

In another embodiment of the invention, the composition may comprise hyaluronic acid as a carrier. Additionally, the treatment method may also be combined with injections of hyaluronic acid combined with certain growth factors. The hyaluronic acid is absorbed in the dermis or the hypodermis with time, thereby providing time release of the growth factor in the composition into the dermis or the hypodermis. Further, hyaluronic acid interacts with CD44 on the mesenchymal stem cells surface.

As described above, hyaluronic acid may be used as a cosmetic dermal filler, which may be used alone to add volume to minimize wrinkles and lines in the face. In the present invention, hyaluronic acid is used as a carrier in the injectable composition of pluripotent cells. When the injectable composition comprises growth factors, the hyaluronic acid also allows for time release of the growth factors into the dermis or the hypodermis. Various forms of commercially available hyaluronic acid, such as non-animal stabilized hyaluronic acid and hyaluronan, can be used for the purpose of the present invention. In an exemplary embodiment, Restylane® and Perlane®, the non-animal stabilized hyaluronic acid, manufactured by Q-med, Seminariegatan, Uppsala, can be used. In the injectable the hyaluronic acid is in a form of gel. The hyaluronic acid gel, such as Restylane® and Perlane® gel particles, is re-absorbed slowly in the injection sites. As the gel breaks down by hydrolysis, water takes its place. The less concentrated the gel becomes, the more water it is able to bind. When totally absorbed, the gel disappears unnoticed from the body. With different hyaluronic acid concentrations and gel particle sizes, the rate of absorption of the gel in the injection sites can be different, hence, the rate of releasing the growth factors in the injectable composition can be different. In this aspect, therefore, hyaluronic acid gel functions as a time release carrier, or a delivery vehicle of the growth factors in the instant injectable composition, which gradually releases the growth factors with time into the dermis or the hypodermis that receives the injection.

### Growth Factors

In one embodiment, the injectable composition comprises stem cells, at least one growth factor, and a suitable carrier. Growth factors are proteins that acts as a signaling molecule between cells (like hormones) that attaches to specific receptors on the surface of a target cell and promote proliferation, differentiation and maturation of cells. Growth factors signify a positive effect on cell growth and cellular differentiation. The growth factors suitable for the purpose of the present invention include, but are not limited to, insulin, insulin-like growth factor, thyroid hormone, fibroblast growth factor, estrogen, retinoic acid, or combination thereof. The stem cell/growth factor compositions of the present invention may be formulated with and without hyaluronic acid.

Suitable thyroid hormone includes, but is not limited to, triiodothyronine (T³), thyroxine (T⁴), or combination thereof. Liothyronine is a synthetic form of the natural triiodothyronine (T³) and is available as the sodium salt. The empirical formula of liothyronine sodium is C₁₅H₁₃I₃NNaO₄ and it has a molecular weight of 672.96. Levothyroxine sodium is a synthetic form of the natural thyroxine (T⁴), which is identical to that produced in the human thyroid gland. Levothyroxine sodium has an empirical formula of C₁₅H₁₀I₄N NaO₄.H₂O, molecular weight of 798.86. In a preferred embodiment, a combination of liothyronine sodium and levothyroxine sodium is used in the injectable composition for stimulating preadipocytes to induce accelerated differentiation into fat cells.

Estrogens are a group of steroid compounds that function as the primary female sex hormone in human body. The three naturally occurring estrogens are estradiol, estriol and estrone. Suitable estrogens for the purpose of the present invention include, but are not limited to, estriol, estradiol, estrone or combination thereof. In a preferred embodiment, estriol is used for stimulating collagen or elastin production in the dermis. Growth factors may be introduced into the hyaluronic acid and stem cells to create a more potent environment for the promotion, differentiation and maturation of the cells of the dermis and of the administered stem cells. The growth factors can be used for the method of the present invention include insulin, an insulin-like growth factor, a thyroid hormone, a fibroblast growth factor, an estrogen, retinoic acid, or combination thereof.

In one embodiment, the injectable composition comprises at least one (e.g., 1, 2, 3, 4, 5, 6, 7, 8, 9, 10, or more) growth factor(s), and optionally hyaluronic acid. In one preferred embodiment, the injectable stem cell composition comprises an estrogen and hyaluronic acid as the carrier.

In another preferred embodiment, the injectable stem cell compositions comprises triiodothyronine, thyroxine, insulin and hyaluronic acid. In some embodiments, the composition further comprises retinoic acid. This composition is preferably used for injection into the hypodermis to stimulate fat cell production. Preferably, liothyronine sodium and levothyroxine sodium, the synthetic forms of triiodothyronine and thyroxine, respectively, are used. The concentration of liothyronine sodium can be in a range from 2 μg to 30 μg per 100 ml of the injectable composition, and preferably from 5 μg to 20 μg per 100 ml of the injectable composition. The concentration of levothyroxine sodium can be in a range from 10 μg to 60 μg per 100 ml of the injectable composition, and preferably from 20 μg to 40 μg per 100 ml of the injectable composition. The concentration of insulin can be in a range from 5 units to 40 units per 100 ml of the injectable stem cell composition, and preferably from 15 units to 30 units per 100 ml of the injectable stem cell composition. The concentration of hyaluronic acid can be in a range from 0.5 g to 3 g per 100 ml of the injectable stem cell composition, and preferably from 1.5 g to 2.5 g per 100 ml of the injectable stem cell composition. The concentration of retinoic acid can be in a range from 5 mg to 25 mg per 100 ml of the injectable stem cell composition, and preferably from 10 mg to 20 mg per 100 ml of the injectable stem cell composition.

Furthermore, the stem cell compositions can further comprise deuterium reduced water and/or dimethylaminoethanol. Dimethylaminoethanol is a precursor to acetylcholine and is used herein to enhance the muscle tone on the face or selected areas of the body. In the injectable composition of the present invention, the concentration of dimethylaminoethanol can be in a range from 2 g to 20 g per 100 ml of the injectable composition, and preferably from 5 g to 15 g per 100 ml of the injectable composition.

Furthermore, the composition can also comprise deuterium reduced water, which functions as an anticarcinogenic agent. The term “deuterium-reduced water” used herein means an aqueous fluid having a deuterium concentration substantially below a naturally occurring deuterium level in water, more specifically, having a deuterium level in a range from about 0.1 ppm to about 110 ppm. The deuterium-reduced water can be produced by distillation or electrolysis, as described in U.S. Pat. Nos. 5,855,921 and 5,788,953, which are hereby incorporated by reference in their entirety. Using electrolysis, the deuterium concentration of water can be reduced down to 30-40 ppm and further reduced to 6-20 ppm by a further electrolysis. Using distillation, the deuterium concentration of water can be reduced down to 20-30 ppm and further reduced to 1-10 ppm by further increasing the plate number and/or repeating the distillation process. To produce a large volume of water, this deuterium depleted water can be mixed with regular water in a predetermined proportion to obtain a water which has the deuterium concentration from about 80 to about 110 ppm, which is substantially lower than the natural occurring level of deuterium in water. In the injectable composition of the present invention, the deuterium reduced water can be a part of the medium, and the concentration of the deuterium reduced water can be in a range from 10 ml to 40 ml per 100 ml of the injectable composition, and preferably from 15 ml to 30 ml per 100 ml of the injectable composition. However, it should be understood that the injectable composition can also be prepared using the regular double distilled water as a medium.

### Adipocytes

In another embodiment of the invention, the injectable compositions of the present invention may further comprise adipocytes or adipocyte progenitor cells. In some embodiments, early adipocyte progenitor cells (Lin⁻:CD29⁺:HCD34⁺:HSca-1⁺:HCD24⁺) are preferred. The adipocytes can function in their traditional role in face lifts by acting as padding; however, the adipocytes are also capable of creating an environment that allows the injected pluripotent cells themselves to commit to becoming adipocytes.

Bone marrow adipogenesis is a normal physiologic process in all mammals and mesenchymal stem cell is the marrow precursor for adipocytes as well as osteoblasts. PPARγ is a measurable differentiation factor for entrance into the fat lineage.

According to another preferred embodiment, there is provided compositions and methods for enhancing engraftment of the stem cells (e.g., PBSC) by co-administering preadipocytes or adipocytes. The preadipocytes or adipocytes may be primary autologous preadipocytes or adipocytes. In some embodiments, the preadipocytes or adipocytes may be obtained by culturing stem cells or fibroblastic cells in vitro under conditions to promote the growth and differentiation of the cells into preadipocytes or adipocytes. Methods for obtaining cellular populations of preadipocytes or adipocytes are well known in the art.

Alternatively, populations of PBSCs (e.g., MSCs), preadipocytes and/or adipocytes may be obtained and sorted from cells collected from a donor. For example, cells collected from the peripheral blood of a subject may generally comprise a comprehensive mixture of cells. That is, there exist a mixture of stem cells, partially differentiated cells (e.g., progenitor cells or fibroblasts), and functional cells (i.e., terminally differentiated cells). According to the general treatment method described herein, an individualized mixture of cells may be generated such as to provide a cellular therapy mixture specific for the needs of a subject. The comprehensive mixture of cells obtained such as through an apheresis process may be characterized, sorted, and segregated into distinct cell populations. Cell markers such as stem cells markers or tissue specific markers may be used to phenotypically characterize the populations of cells collected from the peripheral blood. Using these markers, it is possible to segregate and sort on the basis of cell type. The mixture of cells is thus transformed into populations of cells, which may be broadly classified into two portions: a stem cell portion and a non-stem cell portion. The non-stem cell portion may further be classified into a progenitor cell or fibroblast portion and a function cell or fully differentiated cell portion. Once the peripheral blood cellular mixture is sorted, the stem cell and non-stem cell portions may be cryopreserved and stored separately. In this manner, a library or repository of distinct cell populations from a subject may be created. Alternatively, stem cell and non-stem cell portions may the cryopreserved together and then sorted and separated prior to use.

The types of cell populations that may be generated in this manner include any population of a cell type that developed from a germ layer (i.e., endoderm, mesoderm, and ectoderm). These include, but are not limited to, peripheral blood stem cells, hematopoietic progenitor or differentiated cells, adipocyte progenitor or differentiated cells, neural progenitor or differentiated cells, glial progenitor or differentiated cells, oligodendrocyte progenitor or differentiated cells, skin progenitor or differentiated cells, hepatic progenitor or differentiated cells, muscle progenitor or differentiated cells, bone progenitor or differentiated cells, mesenchymal stem or progenitor cells, pancreatic progenitor or differentiated cells, progenitor or differentiated chondrocytes, stromal progenitor or differentiated cells, cultured expanded stem or progenitor cells, cultured differentiated stem or progenitor cells, or combinations thereof.

The collected stem cells and/or progenitor cells also may be expanded using an ex-vivo process. For example, it may be necessary to expand and propagate a population of stem cells, partially differentiate stem cells to achieve a population of tissue specific progenitor cells (e.g., adipocyte progenitor cells or preadipocytes), or to differentiate stem cells or progenitor cells into fully functional cells (e.g., adipocytes). A variety of protocols have been developed for the enrichment of such populations and are well known in the art. Any known protocol for the expansion or differentiation of stems cells or progenitor cells may be employed. For example, strategies employed may include culturing stem cells or progenitor cells: with or without different cocktails of early and late growth factors; with or without tissue specific growth or differentiation factors; with or without serum; in stationary cultures, rapid medium exchanged cultures or under continuous perfusion (bioreactors); and with or without an established cell feeder layer. In order to achieve maximal ex-vivo expansion of stem cells the following general conditions should be fulfilled: (i) differentiation should be reversibly inhibited or delayed and (ii) self-renewal should be maximally prolonged. Similarly, following cell expansion, it is important to have methods to induce differentiation of the expanded cell population, so as to covert the expanded cell population to mature functional cells or tissue.

The cell populations of the various cells types may then be combined, recombined, or compounded into a cellular therapy mixture of cells appropriate for treating the subject and/or rejuvenating the skin of a subject (e.g., combination of stem cells, adipocytes, and/or adipocyte progenitor cells or fibroblasts). A combination of stem cells, tissue specific progenitor cells, and optionally functional cells is thought to enhance the engraftment of the stem cells.

Accordingly, in one embodiment, the present invention provides methods and products for using an autologous mixture of stem cells, progenitor cells, and optionally functional cells to enhance engraftment of stem or progenitor cells. This cellular therapy product may comprise: from about 10% to about 90% peripheral blood stem cells, about 10% to about 80% peripheral blood stem cells, about 10% to about 60% peripheral blood stem cells, or about 10% to about 40% peripheral blood stem cells; and from about 10% to about 90% non-stem cells, from about 20% to about 90% non-stem cells, from about 40% to about 90% non-stem cells, from about 60% to about 90% non-stem cells. The non-stem portion may optionally comprise from about 5% to about 50% functional cells, about 5% to about 40% functional cells, about 5% to about 30% functional cells, about 5% to about 20% functional cells, or about 5% to about 10% functional cells.

Accordingly, in one embodiment, the present invention provides methods and products for using an autologous mixture of stem cells, progenitor cells, and optionally functional cells to enhance engraftment of stem or progenitor cells. This cellular therapy product may comprise: from about 10% to about 90% stem cells, about 10% to about 80% stem cells, about 10% to about 60% stem cells, or about 10% to about 40% stem cells; and from about 10% to about 90% non-stem cells, from about 20% to about 90% non-stem cells, from about 40% to about 90% non-stem cells, from about 60% to about 90% non-stem cells. The non-stem portion may optionally comprise from about 5% to about 50% functional cells, about 5% to about 40% functional cells, about 5% to about 30% functional cells, about 5% to about 20% functional cells, or about 5% to about 10% functional cells.

A suitable example of the cellular therapy product described above is the autologous mixture of PBSCs (e.g., MSCs), adipocyte progenitor cells, and optionally adipocytes. Methods for stimulating the differentiation of stem cells and preadipocytic cells into adipocytes are described in U.S. Pat. No. 5,728,739, U.S. Pat. No. 5,827,897, and U.S. Publication No. 2010/0015104, the contents of which are incorporated by reference in their entireties.

According to another preferred embodiment, there is provided a method of treating a patient in need thereof comprising administering to a subject an autologous mixture of stem cells, progenitor cells, and optionally functional cells. For example, the present invention is useful to enhance the effectiveness of stem cell and adipocyte progenitor cell engraftment for use in restoring age related tissue loss in the face and selected areas of the body.

Stem cells used to generate preadipocytes or adipocytes may be obtain as described herein, e.g., from PBSC, adipose tissue, or from bone marrow from a human or animal donor. Autologous stem cells or progenitor cells are preferred. Bone marrow can be obtained by any method known to a person of ordinary skill in the art. For example, bone marrow may be removed from a patient by penetration and aspiration of the marrow cavity of the iliac crest, sternum, or other marrow cavity. Bone marrow may also be obtained from human donors undergoing bone resection or exposure of the marrow cavity for other purposes. Bone marrow from research animals or from cadaveric donors may be harvested by dissection of the femur or other bone, excision of the distal ends of the bone, and flushing the marrow cavity into a receptacle.

Bone marrow samples may optionally then be washed to remove contaminants such as bone spicules and medullary adipose, lysed to remove red blood cells, or subjected to differential density sedimentation or other approach that separates adipogenic cells (MSC) from some or all hematopoietic cells. Antibody-mediated positive or negative selection, cell adhesion, and cell culture, may also be applied in enrichment of adipogenic cells.

Cells capable of differentiating into adipocytes may be obtained from tissues other than adipose tissue and bone marrow. For example, enzymatic digestion of skin, blood vessels, or skeletal muscle fragments has been shown to yield cell populations with adipogenic potential. Embryonic stem cells also possess adipogenic potential (Dani, et al., 1997, J. Cell Sci. 110(Pt 11): 1279-85, incorporated herein by reference) and may be generated by means that are known in the art and applied in the present invention.

According to some embodiments, there is provided methods for culturing stem cells (e.g., autologous stem cells) under conditions to promote adipogenesis. Methods by which cells capable of participating in adipogenesis might be cultured are well known in the art. For example, Katz, et al. (U.S. Pat. No. 6,777,231, incorporated herein by reference), have described methods of culturing adipose tissue-derived stem cells. Similarly, Hamilton et al (U.S. Pat. No. 5,783,408, incorporated herein by reference) have described means for culturing preadipocytes for use in drug screening. Pittenger, et al. (U.S. Pat. No. 5,827,740, incorporated herein by reference) have described means by which mesenchymal stem cells may be cultured and induced to undergo adipogenesis. Dani et al. (1997, J. Cell Sci. 110(Pt 11): 1279-85, incorporated herein by reference) have described means for culturing embryonic stem cells and inducing the cells to undergo adipogenesis. In general, these methods apply a basal cell culture medium to expand cell numbers followed by induction of adipogenesis by culturing cells in medium containing agents such as dexamethasone, 3-isobutyl-1-methylxanthine, activators of peroxisome proliferator-activated receptor gamma gene product, and insulin. Activators of peroxisome proliferator-activated receptor gamma are well known in the art. See e.g., U.S. Pat. No. 5,994,554, incorporated herein by reference.

In some embodiments, stem cells (e.g., autologous MSCs) are induced to differentiate into the adipogenic lineage by employing a glucocorticoid; insulin; and at least one compounds which inhibit degradation of cAMP (e.g., indomethacin). In a particularly preferred embodiment, the indomethacin is used in conjunction with methyl isobutylxanthine.

Stem cells or progenitor cells may also be genetically modified to express genes to induce adipogenesis using methods known to those of skill in the art. Examples of genetic modifications that could be made to stem cells generated according to the present invention include the introduction of mutant or polymorphic receptors and other molecules associated with adipogenesis, obesity or diabetes, for example, insulin receptor, Peroxisome Proliferator-Activated Receptor Gamma (PPARγ), aP2, leptin, and adiponectin). For example, the PPARγ gene has several polymorphisms in the normal population. Two exemplary PPARy polymorphisms result in Prol115Gln and Pro12Ala.

Using the methods of the present invention it is possible to introduce genes encoding polymorphic proteins of interest into cells capable of undergoing adipogenesis and then generating preadipocytes and adipocytes for use in the compositions of the present invention.

### Injection Methods

In a further aspect, the present invention is directed to the methods of using the compositions of the present invention for restoring age related tissue loss in the face and selected areas of the body. In some embodiments, the present invention provides for methods for topically administering or injecting the compositions of the present invention to the dermis or hypodermis of a subject in need thereof. The subject is administered the stem cell compositions of the present invention over one or more sessions within a treatment period (e.g., 1 to 10 weeks). The number of session may be from 1 to 10 (e.g., 1, 2, 3, 4, 5, 6, 7, 8, 9, and 10). The frequency of administration may range from 1, 2, or 3 times a week. Preferably, three sessions of injections are administered sequentially. In some embodiments, the three sessions of injections are administered within a one week to three month time period (e.g., 1, 2, 3, 4, 5, 6, 7, 8, 9, 10 11, or 12 week time period), and preferably within a month of the first treatment.

In one embodiment, the treatment method comprises the steps of providing a composition comprising stem cells and a carrier; and injecting the composition into the dermis and/or the hypodermis at one or more areas of the face, or selected areas of the body of a person, thereby restoring age-related tissue loss in the face, or the selected areas of the body of the person.

The suitable areas, or injection sites, include, but are not limited to, the periorbital area, the lips, the malar area, the nasolabial folds, the labio-mandibular folds, and selected areas of the body, such as the neck, and the hands. The periorbital area includes the eyelids and surrounding areas including the eyebrows, bony eye socket and rims, cheeks and forehead. Malar area includes cheek or the side of the head. The nasolabial folds are the deep folds which run from the side of the nose to the corner of the mouth. The labiomandibular folds are the folds between the corner of the mouth and the jawbone.

For stem cell-based treatments, a stem cells are preferably collected from an autologous or heterologous human or animal source. An autologous animal or human source is more preferred. Stem cell compositions are then prepared and isolated as described herein. To introduce or transplant the stem cells and/or compositions comprising the stem cells according to the present invention into a human or animal recipient, a suspension of mononucleated cells is prepared. Such suspensions contain concentrations of the stem cells of the invention in a physiologically-acceptable carrier, excipient, or diluent. For example, suspensions of stem cells for administering to a subject preferably comprise 10⁵ to 10¹² cells/ml in a sterile solution of complete medium modified to contain the subject's serum, as an alternative to fetal bovine serum. Alternatively, stem cell suspensions may be in serum-free, sterile solutions, such as cryopreservation solutions. Enriched stem cell preparations may also be used. The stems suspensions may then be introduced e.g., via injection, into one or more sites of the donor tissue.

Concentrated or enriched cells may be administered as a pharmaceutically or physiologically acceptable preparation or composition containing a physiologically acceptable carrier, excipient, or diluent, and administered to the tissues of the recipient organism of interest, including humans and non-human animals. The stem cell-containing composition may be prepared by resuspending the cells in a suitable liquid or solution such as sterile physiological saline or other physiologically acceptable injectable aqueous liquids. The amounts of the components to be used in such compositions can be routinely determined by those having skill in the art.

For injectable administration, the composition is in sterile solution or suspension or may be resuspended in pharmaceutically- and physiologically-acceptable aqueous or oleaginous vehicles, which may contain preservatives, stabilizers, and material for rendering the solution or suspension isotonic with body fluids (i.e. blood) of the recipient. Non-limiting examples of excipients suitable for use include water, phosphate buffered saline, pH 7.4, 0.15 M aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as the routes of administration used, are determined on an individual basis, and correspond to the amounts used in similar types of applications or indications known to those of skill in the art.

The treatment can be performed without local or general anesthesia. Typically, a patient is placed in a treatment room, the injections are given using a needle (e.g., 30 gauge needle) with a syringe (e.g., 3 to 5 cc syringe). In some embodiments, the hypodermis of a specific area is treated with 0.01 to 0.3 ml of the compositions of the present inventions (e.g., 0.01, 0.02, 0.03, 0.04, 0.05, 0.06, 0.07, 0.08, 0.09, 0.1, 0.2, 0.3 ml). Preferably, each dose comprises at least 1×10⁵ total nucleated cells (e.g., at least on the order of 10¹¹, 10¹⁰, 10⁹, 10⁸, 10⁷, 10⁶, or 10⁵ totalnucleated cells). In some embodiments, each dose comprises at least 1×10⁵ pluripotent stem cells (e.g., at least on the order of 10¹¹, 10¹⁰, 10⁹, 10⁸, 10⁷, 10⁶, or 10⁵ total nucleated cells). In some embodiments, each dose comprises at least 1×10⁵ MSCs (e.g., at least on the order of 10¹¹, 10¹⁰, 10⁹, 10⁸, 10⁷, 10⁶, or 10⁵ totalnucleated cells). In some embodiments, each dose comprises at least 1×10⁵ CD34+ cells (e.g., at least on the order of 10¹¹, 10¹⁰, 10⁹, 10⁸, 10⁷, 10⁶, or 10⁵ total nucleated cells). In some embodiments, the compositions are delivered to the at various facial areas at the hypodermis level.

In a further embodiment, the method comprises injecting a first composition of the present invention into the dermis at one or more areas of the face or selected areas of the body of a person; and injecting the second composition into the hypodermis at the same areas of the face or selected areas of the body; thereby restoring age related tissue loss in the face, neck or hands of the person. The first and the second compositions may be the same or different. For example, the first composition may comprise stem cells in a suitable carrier and the second composition may comprise stem cells in a suitable carrier with growth factors and/or adipose cells.

The two layer treatment may be performed without local or general anesthesia. In this procedure, a needle (e.g., an adjustable 30 gauge needle) with a syringe (e.g., 3 to 5 cc syringe) can be used to inject the first composition at selected areas at the dermis level first. With, for example, 0.1 ml increments, the dermis of a specific area is treated. Upon completing the dermal injection, the adjustable 30 gauge needle is adjusted so that the needle length is longer for injection in the subcutaneous tissue (hypodermis). Alternatively, the needle can be changed to a longer one for subcutaneous injection. The second composition is then injected in the same selected areas, at the hypodermis level. Preferably, three sessions of injections are administered sequentially within the time periods disclosed herein.

Stem cells and compositions comprising stem cells of the present invention can be used to repair, treat, or ameliorate various aesthetic or functional conditions (e.g., defects) through the augmentation. The stem cells of the present embodiments may provide an important resource for rebuilding or augmenting damaged or lost tissue. In addition, such stem cells and compositions thereof can be used for augmenting soft tissue not associated with injury by adding bulk to a soft tissue area, opening, depression, or void in the absence of disease or trauma, such as for “smoothing”. Multiple and successive administrations of stem cells are also embraced by the present invention.

The stem cells and/or non-stem cells (e.g., preadipocytes and/or adipocytes) are preferably administered in suspension with a suitable carrier as described herein. In some embodiments the cells may be loaded onto small biocompatible/biodegradable matrixes or scaffolds (for example beads or microbeads). Cells may be injected in a simple aqueous solution such as physiologic saline or in an injectable hydrogel such as collagen-based hydrogel. Cells may be delivered on a bead-like or particulate scaffold using injection provided that a sufficiently large gauge needle is used such that the beads do not block the needle or that the application of injection force does not apply a degree of shear force to the beads or cells resulting in significant reduction of the integrity of the scaffold or viability of the cells.

It should be understood that although the method of the present invention has been disclosed with a general focus on the restoration of age related tissue loss in the face, the method can be used in other selected areas of the body, such as the neck, and hands, for the same purpose.

### Microdermabrasion

The invention is further defined by the use of microdermabrasion, which is performed just prior to the injection of stem cells. Microdermabrasion is a procedure that involves the skin being blasted by aluminum oxide crystals, baking soda, salt or corn cob granules to remove the stratum corneum (top) layer of the skin, which includes dead skin cells. Microdermabrasion promotes the production of new cells in the basal layer of the dermis, which creates an environment that is beneficial to newly introduced pluripotent cells. Essentially the mircodermabrasion results in the need for new cells that causes an upregulation of many growth signals that reinforce the commitment of pluripotent cells to differentiate.

The following example is illustrative of the invention and is in no way to be interpreted as limiting the scope of the invention, as defined in the claims. It will be understood that various other ingredients and proportions may be employed, in accordance with the proceeding disclosure.

### Stem Cell Collection Process

Stem cells may be found in the insides of long bones (legs, hips, sternum etc.) and comprise the “bone marrow”. These stem cells may leave the bone marrow and circulate in the blood stream. The physical steps of collecting stem cells may comprise those steps known in the art. Stem cells comprise approximately 0.1-1.0% of the total nucleated cells as measured by the surrogate CD34+ cells.

According to a preferred embodiment, the stem cells may be collected by an apheresis process, which typically utilizes an apheresis instrument. The apheresis instrument looks very much like a dialysis machine, but differs in that it is a centrifuge while a dialysis machine uses filtration technology. Stem cell collection can be accomplished in the privacy of the donors own home or in a collection center. Blood is drawn from one arm then enters the apheresis instrument where the stem cells are separated and collected. The rest of the whole blood is then returned to the donor. A registered nurse (RN) places a needle into both arms of the subject in the same manner as a routine blood collection. The RN then operates the apheresis instrument that separates the blood elements (red cells, white cells, plasma) collecting the stem cells and returning the rest of the whole blood to the donor. The collection of stem cells requires approximately 2-4 hours during which the subject is relaxing and watching a movie. Shortly after the apheresis collection, the bone marrow releases more stem cells into the blood stream to replace the harvested stem cells. The amount of stem cells collected is a very small fraction of a person's stem cells. In a healthy individual, the stem cells can rapidly multiply and replace the lost stem cells. Thus, the procedures of the invention does not deplete the body of stem cells. Many hundreds of thousands of apheresis collections take place each year for platelets, red cells, plasma and stem cells. It has been shown to be safe and effective technology.

According to a preferred embodiment, there is provided a method for collecting autologous adult stem cells from a human subject; collecting adult stem cells from peripheral blood pre-disease human subject using an apheresis process; at the time of collection, earmarking the collected cells for use by the human subject; and preserving the collected cells to maintain the cellular integrity of the cells.

Collection may be performed on any person. Furthermore, collection may involve one or more collecting steps or collecting periods. For example, collection (e.g., using an apheresis process) may be performed at least two times, at least three times, or at least 5 times on a person. During each collecting step, the number of total nucleated cells collected per kilogram weight of the person may be one million (1×10⁶) or more, two million or more, three million or more, or 5 million or more.

### DEFINITIONS

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only not intended to be limiting. Other features and advantages of the invention will be apparent from the following detailed description and claims.

For the purposes of promoting an understanding of the embodiments described herein, reference will be made to preferred embodiments and specific language will be used to describe the same. The terminology used herein is for the purpose of describing particular embodiments only, and is not intended to limit the scope of the present invention. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth. Thus, for example, a reference to “a host cell” includes a plurality of such host cells, and a reference to “an antibody” is a reference to one or more antibodies and equivalents thereof known to those skilled in the art, and so forth. Further, the use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the term “stem cell” refers to a cell with the ability to proliferate and to differentiate towards cells of more than one specialized cell type. For example, a cell that is capable of proliferating and of differentiating into cells with characteristics of adipocytes and into cells of the bone and/or of muscle fulfills this definition.

It should be understood that there is a distinction between a “hematopoietic stem cells” or “hematopoietic pluripotent stem cell” and a “stem cell collected from the hematopoietic system.” A “hematopoietic stem cells” or “hematopoietic pluripotent stem cell” is a stem cell that by differentiation, and division, can repopulate the various lineages of the hematopoietic system. Hematopoietic stem cells (HSC) are stem cells and the early precursor cells which give rise to all the blood cell types that include both the myeloid (monocytes and macrophages, neutrophils, basophils, eosinophils, erythrocytes, megakaryocytes/platelets and some dendritic cells) and lymphoid lineages (T-cells, B-cells, NK-cells, some dendritic cells). A hematopoietic pluripotent stem cell does not have to be collected from the hematopoietic system. For example, hematopoietic stem cells may be collected from gut, spleen, kidney or ovaries—tissues that are not part of the hematopoietic system.

Conversely, a “stem cell collected from the hematopoietic system,” such as the “peripheral blood stem cells” or “PBSC” collected, for example, by an apheresis process of may be a stem cell for all tissue types in the body. That is, a stem cell collected by apheresis process may be any stem cell, such as a neural stem cell, an adipose tissue stem cell, a liver stem cell, a muscle stem cell, or a hematopoietic stem cell, etc. Thus, a stem cell collected from the peripheral blood may give rise to any lineage of cells in a mammalian body, such as, for example, a neural stem cell, an adipose tissue stem cell, a liver stem cell, a muscle stem cell, or a hematopoietic stem cell, etc.

As used in the context of the present invention described herein, the term “donor” or “subject” refers to a person or a animal from whom stem cells are collected. The stem cells may be used in an autologous transfer for future treatment of that same donor or subject. The terms “donor” or “subject” refers to all animals, in particular vertebrates, for which the collection of peripheral blood is possible. Examples of such vertebrates are mammals which includes human and commercially valuable livestock and research animals such as horses, cows, goats, rats, mice, rabbits, pigs, and the like. An example of such a mammal is a human such as a human infant, child, or adult. For ease of discussion in this patent application, we use a human being (identified as a person, a patient or a donor) as a non-limiting example of such an animal. According to preferred embodiments, the subject is a human.

As used herein, the term “adipocyte” refers to a cell that is specialized to synthesize and store fat. This term includes adipocytes with the properties representative of those present within white fat, yellow fat, and brown fat.

As used herein, the term “adipose tissue” refers to a tissue that contains adipocytes that may or may not be accompanied by stromal cells, blood vessels, lymph nodes, tissue macrophages, and other cells and structures. The term includes tissue that is commonly referred to in the art as white adipose tissue (or white fat), to brown adipose tissue (or brown fat), and to yellow adipose tissue (or yellow fat). Adipose tissue is normally found in multiple sites within the body including, but not limited to subcutaneous adipose, visceral adipose, omental adipose, perirenal adipose, scapular adipose, inguinal adipose, adipose surrounding lymph nodes, medullary adipose, bone marrow adipose, pericardial adipose, retro-orbital adipose, and infrapatellar adipose. In the context of the present invention the term “adipose tissue” also refers to tissue that contains adipocytes or preadipocytes, said adipocytes and/or preadipocytes being derived from implantation of donor cells capable of differentiating into preadipocytes and/or adipocytes. The term further includes tissue that does not yet contain adipocytes but which is a precursor or anlage of such tissue.

As used herein, the term “preadipocyte” refers to a cell capable of differentiating into an adipocyte. In particular, a preadipocyte contains little or no stored fat.

As used herein, the term “adipogenesis” is a collective term that refers to the processes by which adipocytes are formed. The term applies both to the entire process by which an undifferentiated cell differentiates into an adipocyte and to steps within this process. For example, the term adipogenesis can apply to the maturation of a preadipocyte into an adipocyte, to the process by which precursors of preadipocytes (for example, stem cells) differentiate into preadipocytes, to combinations of such processes, and to subsets of the process by which a stem cell differentiates into an adipocyte.

### EXAMPLES

It is understood that modifications which do not substantially affect the activity of the various embodiments of this invention are also provided within the definition of the invention provided herein. Accordingly, the following examples are intended to illustrate but not limit the present invention. While the claimed invention has been described in detail and with reference to specific embodiments thereof, it will be apparent to one of ordinary skill in the art that various changes and modifications can be made to the claimed invention without departing from the spirit and scope thereof. Thus, for example, those skilled in the art will recognize, or be able to ascertain, using no more than routine experimentation, numerous equivalents to the specific substances and procedures described herein. Such equivalents are considered to be within the scope of this invention, and are covered by the following claims.

### Example 1

### Protocol for Skin Rejuvenation with Autologous Adult Stem Cells

The skin is cleansed with 70% alcohol. An Emed, Inc. SilkPeel™ treatment is then administered to remove the superficial epidermal barrier and gross bacteria. The skin is then vacuumed using a standard dermatological vacuum set on high. The skin is then exposed to a Omnilux blu lamp (wavelength 415 nm) for 15 minutes to kill residual bacteria. This is followed by exposure to a Omnilux red lamp (wavelength 633 nm) to open the pores of the skin and to dilate the subdermal plexus to improve stem cell survival.

A second cleansing of the skin with 70% alcohol is performed. At this time, the previously harvested autologous stem cells in 2 vials having approximately 5,000,000 cells per vial, are removed from liquid nitrogen and placed in warm water. Upon thawing the cells are centrifuged for 5 minutes to allow the cells to form a pellet. The DMSO in which the cells have been frozen is removed with a small syringe. The stem cells are suspended in physiological saline.

The stem cells in physiological saline are placed in an Emed bottle port. The machine is set at low vacuum. One vial of the stem cells is suspended in 40 cc of saline, which is used for the Emed infusion technique. The complete face is treated with only one pass using the Emed infusion device; thereby, infusing the stem cells into the superficial dermis. The Emed device simultaneously microabrades and infuses the stem cells into the dermis of the skin.

Upon completion of the Emed dermal infusion, a second pellet of stem cells is suspended in 12 cc of saline and loaded into four 3-cc syringes with an adjustable small needle set at dermal depth. The stem cells are injected intradermally at a depth of 2 mm at the site of deep wrinkles, rhytids and crow's feet, as well as the nasolabial folds (laugh lines), forehead wrinkles, etc. This varies on each patient based on their wrinkle configuration.

A petroleum occulusive dressing, e.g., AQUAPHOR, is placed on the full face and the patient remains in the lying position for 20 minutes at rest.

This protocol is repeated one week after the initial treatment. Thereafter, the protocol is performed every two months for one year for a total of 8 treatments to optimize the superficial skin rejuvenation process.

Each evening and morning following the first treatment, a topical DNA cream is applied to the skin. This continues as an ongoing skin restoration process for one year. The cream stimulates collagen and evens out skin tone and color.

### Example 2

### Screening Study to Test the Adipogenesis Effect of Two Specific Growth Factor Formulas on Human Mesenchymal Stem Cells

A screening method was performed to evaluate the adipogenic potential of two different growth factor formulas (GFF-1, GFD-1) on human bone marrow-derived mesenchymal stem cells. The growth factor formulas were embedded in a hyaluronic acid gel matrix to allow for a long (3 month) release of growth factor matrix when used in vivo.

Since the intent was to screen for the effects of each one of the growth factor formulas separately, they were transferred with a sterile pipette to separate culture plates containing human mesenchymal stem cells.

The mesenchymal stem cells were seeded at three different concentrations in E-well tissue culture plates: 10³, 10⁴, and 10⁵ μl.

Results

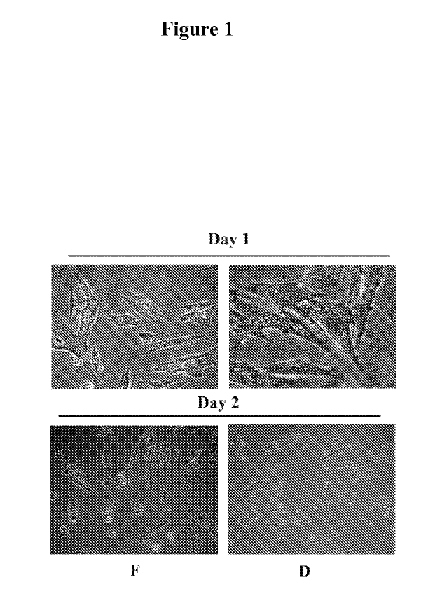

After 1 day, the cells were examined for fat globules. As depicted in the top (rt) and (lt) panels of FIG. 1, >95% of the cells showed evidence of adipogensis. The seeding densities of mesenchymal stem cells did not affect adipogenesis. On day 2, the wells were examined with a lower magnification to better assess the efficiency of differentiation. The results showed no difference from day 1, indicating that adipogenesis was rapid, as depicted in the bottom (rt) and (lt) panel of FIG. 1.

Discussion

The rapid formation of adiposite-like cells is highly unusual, which in normal circumstances take greater than 1 month normally with commercially available differentiation media. Another positive observation was the lack of fibroblasts, which if differentiated from mesenchymal stem cells, could lead to fibrosis. Finally, there was no observed evidence of abnormal cell morphology or cellular malignancy in the short initial study.

What is claimed is:

1. A composition comprising stem cells and hyaluronic acid as a carrier, wherein said stem cells are peripheral blood stem cells, bone marrow-derived stem cells, or mesenchymal stem cells.

2. The composition of claim 1, wherein the stem cell are autologous stem cells.

3. The composition of claim 1, wherein the composition further comprises adipocytes or preadipocytes.

4. The composition of claim 1, wherein the composition further comprises insulin, an insulin-like growth factor, a thyroid hormone, a fibroblast growth factor, an estrogen, retinoic acid, or combination thereof.

5. The composition of claim 1, wherein the thyroid hormone is liothyronine sodium, levothyroxine sodium, or combination thereof.

6. The composition of claim 1, wherein said one or more areas of the face is the peri-orbital area, the lips, the malar area, the nasolabial folds, the labio-mandibular folds, the neck, or the hands.

7. The composition of claim 1, wherein said composition further comprises retinoic acid.

8. The composition of claim 1, wherein said composition further comprises deuterium reduced water.

9. A treatment method for restoring tissue volume in the face or selected areas of the body of a person comprising administering to a human subject a composition comprising stem cells and hyaluronic acid as a carrier, wherein said stem cells are peripheral blood stem cells, bone marrow-derived stem cells, or mesenchymal stem cells, and wherein said composition is administered via injection into the dermis or the hypodermis at one or more areas of the face, thereby restoring tissue volume said selected areas of the body of said person.

10. The treatment method of claim 9, wherein the stem cell are autologous stem cells.

11. The treatment method of claim 9, wherein the composition further comprises adipocytes or preadipocytes.

12. The treatment method of claim 9, wherein the composition further comprises insulin, an insulin-like growth factor, a thyroid hormone, a fibroblast growth factor, an estrogen, retinoic acid, or combination thereof.

13. The treatment method of claim 9, wherein the thyroid hormone is liothyronine sodium, levothyroxine sodium, or combination thereof.

14. The treatment method of claim 9, wherein said one or more areas of the face is the peri-orbital area, the lips, the malar area, the nasolabial folds, the labio-mandibular folds, the neck, or the hands.

15. The treatment method of claim 9, wherein said composition further comprises retinoic acid.

16. The treatment method of claim 9, wherein said composition further comprises deuterium reduced water.
